(19) United States
(12) Patent Application Publication
(10) Pub. No.: US 2024/0306996 A1
(43) Pub. Date: Sep. 19, 2024

(54) WATCH PROTECTION CASE FOR ASSISTING IN DISPLAYING ELECTROCARDIOGRAM

(71) Applicant: Shenzhen Yise Technology Co., Ltd, Shenzhen (CN)

(72) Inventor: Lixin Deng, Shenzhen (CN)

(21) Appl. No.: 18/677,813

(22) Filed: May 29, 2024

(30) Foreign Application Priority Data
Apr. 9, 2024 (CN) 202410422092.6

(51) Int. Cl. A61B 5/00; A61B 5/28; A61B 5/339

(52) U.S. Cl. CPC A61B 5/681; A61B 5/28; A61B 5/339; A61B 2560/0468

(57) ABSTRACT

A watch protection case for assisting in displaying an electrocardiogram is provided, including a case body and a knob component; an accommodating chamber for placing a watch body of a watch is arranged inside the case body; a notch where a detection portion protrudes out is arranged on a bottom wall of the accommodating chamber; a rotary hole is formed in a side wall of the accommodating chamber; the knob component is made of a conductive material, and the knob component is rotatably connected to the rotary hole and can move in an axial direction of the rotary hole; a head end of the knob component is arranged outside; and a tail end of the knob component is arranged in the accommodating chamber, sleeves a crown, and abuts against a titanium electrode, so as to form a signal loop.

### TECHNICAL FIELD

The present disclosure belongs to the technical field of watch accessories, and in particular, to a watch protection case for assisting in displaying an electrocardiogram.

### BACKGROUND

With the development and progress of technology, smartwatches have gradually become indispensable electronic products in daily life. A smartwatch has many functions such as remote unlocking, heart rate detection, and electrocardiogram display. As shown in FIG. 1, when an electrocardiogram display function is used, electrical signals of a fingertip and the wrist are respectively detected through a crown that is covered with a titanium electrode and located on a side edge of a watch body and a detection portion that is covered with silicochromium carbon nitride and located at a bottom of the watch body. A loop is formed according to two acquisition points to record an electrical signal of the heart, and an electrocardiogram is displayed on a touch screen. For users with sinus arrhythmia and atrial fibrillation, this can play a warning and risk avoiding role.

The watch body and the touch screen of the smartwatch are easily scratched during use, so watch protection cases have emerged on the market. A watch protection case can surround the smartwatch to avoid damage caused by collision between the watch body and the touch screen of the smartwatch by relying on a buffering effect. According to the patent No. 2023205699394, it can be seen that a knob component corresponding to a crown may be arranged on side walls of some watch protection cases, to assist a user in operating the smartwatch.

However, in actual use, it is found that the existing knob components do not have an electricity conduction function, so these knob components cannot transmit an electrical signal of a fingertip of a user to the crown, thereby affecting formation of a signal loop. As a result, the electrocardiogram display function of the smartwatch cannot be normally used. This brings a great inconvenience to the user. Therefore, it is in an urgent need for designing a watch protection case that can be used to assist in displaying an electrocardiogram.

### SUMMARY

### (I) Technical Problems to be Solved

The present disclosure provides a watch protection case for assisting in displaying an electrocardiogram. A knob component of the watch protection case has an electricity conduction function to transmit an electrical signal of a fingertip of a user to a crown to form a signal loop, thereby ensuring normal use of an electrocardiogram display function of a smartwatch.

### (II) Technical Solutions

In order to achieve the above objective, the present disclosure provides the following technical solutions:

A watch protection case for assisting in displaying an electrocardiogram includes a case body and a knob component; an accommodating chamber for placing a watch body of a watch is arranged inside the case body; a notch where a detection portion of the watch body protrudes out is arranged on a bottom wall of the accommodating chamber; a rotary hole corresponding to a crown is formed in a side wall of the accommodating chamber; the knob component is made of a conductive material, and the knob component is rotatably connected to the rotary hole and can move in an axial direction of the rotary hole; a head end of the knob component is arranged outside; and a tail end of the knob component is arranged in the accommodating chamber to sleeve a periphery of the crown and abut against a titanium electrode on the crown.

Preferably, the knob component includes a wheel knob and a crown sleeve; the crown sleeve is arranged in the accommodating chamber to sleeve the periphery of the crown and abut against the titanium electrode of the crown; a shaft rod that is rotatably connected to the rotary hole and can move in the axial direction of the rotary hole is arranged on the crown sleeve; the wheel knob is arranged outside and is connected to the shaft rod, so as to control rotation and movement of the crown sleeve; and the crown sleeve, the shaft rod, and the wheel knob are all made of the conductive material.

Preferably, an outer wall of the case body is provided with a convex column; the rotary hole is coaxially arranged in the convex column; the wheel knob is provided with a concave cavity; the wheel knob is coaxially provided with an integrally formed sleeve in the concave cavity; the concave cavity is matched with the convex column; and the sleeve sleeves and fixes the shaft rod.

Preferably, the knob component further includes a flexible contact block; the flexible contact block is in a plane design and is mounted in the crown sleeve; the crown sleeve tightly abuts against the titanium electrode of the crown through the flexible contact block; and the flexible contact block is also made of a conductive material.

Preferably, the crown sleeve, the shaft rod, and the wheel knob are all made of a metal conductive material such as copper, iron, and 316 stainless steel, and the flexible contact block is made of a flexible conductive material such as conductive foam, a conductive adhesive, and conductive cloth.

Preferably, the knob component further includes an elastic body; the case body is provided with a driving chamber on an inner side of the rotary hole; the shaft rod can penetrate through the driving chamber and the rotary hole in sequence; and the elastic body is arranged in the driving chamber and resists against the crown sleeve and a top wall of the driving chamber to drive the crown sleeve to tightly abut against the titanium electrode of the crown.

Preferably, the clastic body is a spring; the spring sleeves the shaft rod; and the two ends of the spring respectively resist against the crown sleeve and the top wall of the driving chamber.

Preferably, the knob component further includes a sealing ring; the case body uses a sealed waterproof design; the case body is provided with a sealing chamber on an outer side of the rotary hole; the shaft rod can penetrate through the rotary hole and the sealing chamber in sequence; the sealing ring is arranged in the sealing chamber and sleeves the shaft rod; and the scaling ring resists and abuts against the shaft rod and an inner wall of the sealing chamber.

Preferably, a plurality of anti-slip slots are arranged in a circular array on an inner wall of the crown sleeve to be tightly matched with a peripheral side wall of the crown.

Preferably, skidproof stripes are arranged on a circumferential side wall of the wheel knob.

Preferably, the knob component further includes a decorative ring; and the decorative ring is mounted on the wheel knob in a sleeving manner.

### (III) Beneficial Effects

According to the watch protection case for assisting in displaying the electrocardiogram, the case body is designed to protect a watch body and a touch screen of a smartwatch to avoid the smartwatch from being damaged by collision. The notch is designed on the case body, so that the wrist can abut against the detection portion to transmit an electrical signal. The knob component is designed to drive the crown, so that a user can operate the smartwatch. As the knob component is made of the conductive material, a fingertip can transmit an electrical signal to the titanium electrode, causing two acquisition points of the smartwatch to form a loop to record an electrical signal of the heart. This ensures that the electrocardiogram display function can be normally used and improves the convenience of use.

### BRIEF DESCRIPTION OF THE DRAWINGS

The accompanying drawings are intended to provide a further understanding of the present disclosure and constitute a part of this specification. The accompanying drawings and embodiments of the present disclosure are used together to explain the present disclosure rather than constituting a limitation on the present disclosure. In the drawings:

FIG. 1 shows a schematic diagram of use of an electrocardiogram display function of a smartwatch;

FIG. 2 shows a schematic diagram of an entire structure of the present disclosure;

FIG. 3 shows a partially schematic structural diagram I of the present disclosure;

FIG. 4 shows a top view of FIG. 3;

FIG. 5 shows a sectional view A-A of FIG. 4;

FIG. 6 shows an enlarged view of the part A in FIG. 5;

FIG. 7 shows a front view of FIG. 3;

FIG. 8 shows a sectional view B-B of FIG. 7;

FIG. 9 shows an enlarged view of the part B in FIG. 8;

FIG. 10 shows a schematic exploded diagram I of FIG. 3;

FIG. 11 shows a schematic exploded diagram II of FIG. 3; and

FIG. 12 shows a schematic exploded diagram of a knob component according to the present disclosure.

In the drawings: 1: case body; 10: accommodating chamber; 11: notch; 12: rotary hole; 13: convex column; 14: driving chamber; 15: sealing chamber; 2: knob component; 21: wheel knob; 211: concave cavity; 212: sleeve; 213: skidproof stripe; 22: crown sleeve; 220: shaft rod; 221: anti-slip slot; 23: flexible contact block; 24: elastic body; 25: sealing ring; 26: decorative ring; 3: watch body; 31: detection portion; 32: crown; 320: titanium electrode; and 33: touch screen.

### DETAILED DESCRIPTION OF THE EMBODIMENTS

The technical solutions in embodiments of the present application are clearly and completely described in the following with reference to the accompanying drawings in the embodiments of the present application. Apparently, the described embodiments are merely some rather than all of the embodiments of the present application. All other embodiments obtained by a person of ordinary skill in the art based on the embodiments of the present disclosure without making creative efforts shall fall within the protection scope of the present disclosure. It can be understood that the drawings are only for reference and illustration purposes and are not intended to limit the present disclosure. The connection relationship shown in the accompanying drawings is only for the sake of clear description and does not limit a connection method.

It should be noted that when an assembly is considered to be “connected” to another assembly, the assembly can be directly connected to the another assembly or there may be an intermediate assembly. Unless otherwise defined, all technical and scientific terms used herein have the same meaning as those commonly understood by a person skilled in the art to which the present disclosure belongs. It should be also noted that unless otherwise specified and limited, the terms “mount”, “connect”, and “connection” should be broadly understood. For example, it can be a fixed connection, detachable connection, integrated connection, mechanical connection, electrical connection, or internal communication between two elements. A person of ordinary skill in the art can understand the specific meanings of the above terms in the present disclosure according to specific situations. Terms used in the specification of the present disclosure herein are merely intended to describe objectives of the specific embodiments, but are not intended to limit the present disclosure.

It should be also noted that in the descriptions of the present disclosure, orientations or positional relationships indicated by the terms “center”, “upper”, “lower”, “left”, “right”, “vertical”, “horizontal”, “inner”, “outer”, and the like are orientations or positional relationships as shown in the drawings, and are only for the purpose of facilitating and simplifying the descriptions of the present disclosure instead of indicating or implying that devices or elements indicated must have particular orientations, and be constructed and operated in the particular orientations, so that these terms are not construed as limiting the present disclosure. In addition, the terms “first”, “second”, and “third” are only for the purpose of description, and may not be understood as indicating or implying the relative importance.

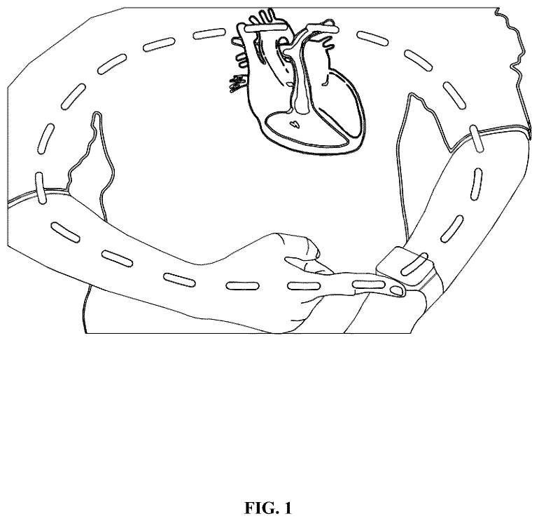

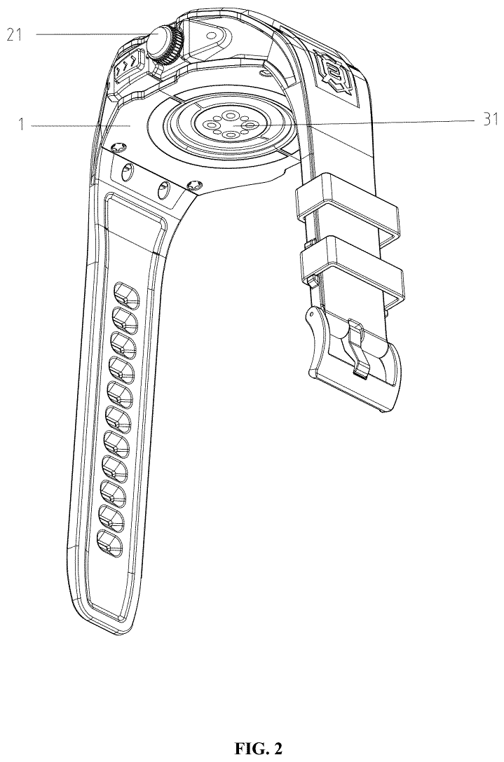

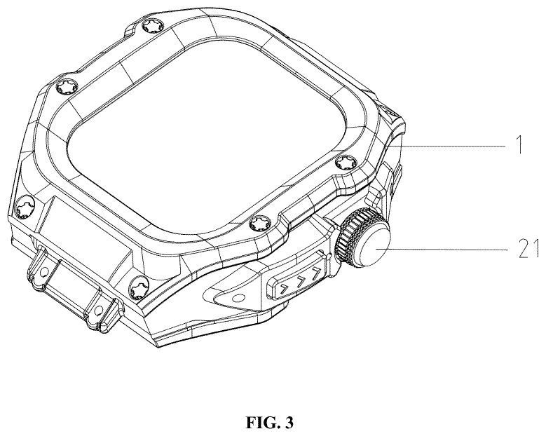

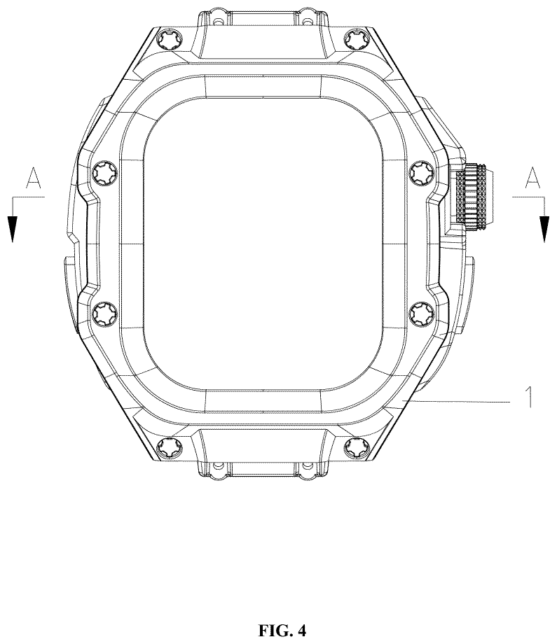

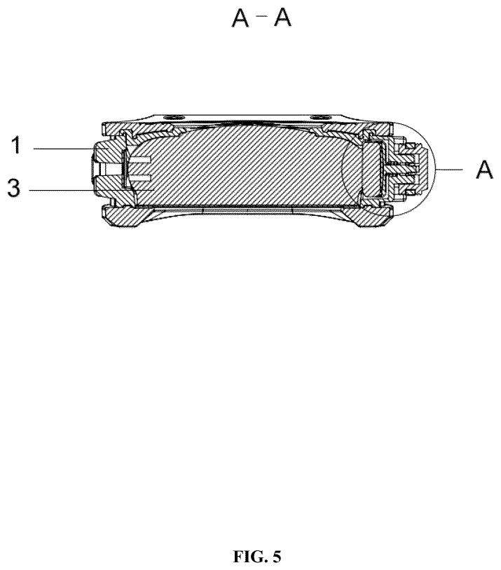

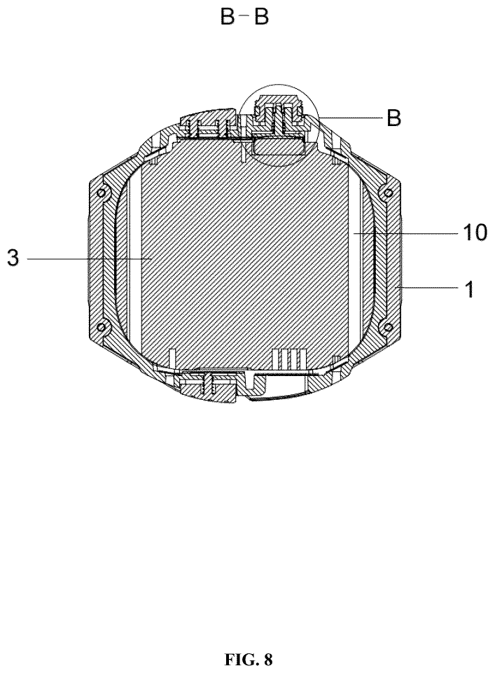

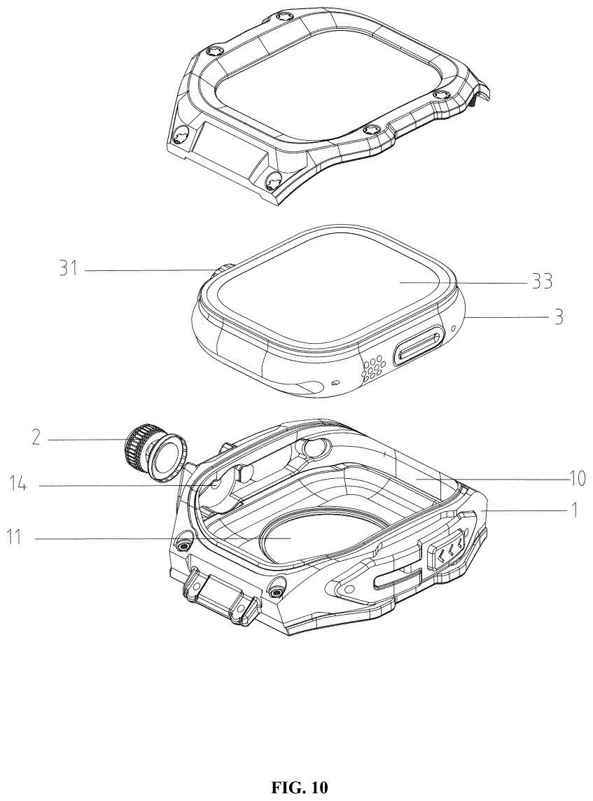

Referring to FIG. 1 to FIG. 10, a watch protection case for assisting in displaying an electrocardiogram includes a case body 1 and a knob component 2; an accommodating chamber 10 for placing a watch body 3 of a watch is arranged inside the case body 1; a notch 11 where a detection portion 31 of the watch body 3 protrudes out is arranged on a bottom wall of the accommodating chamber 10; a rotary hole 12 corresponding to a crown 32 is formed in a side wall of the accommodating chamber 10; the knob component 2 is made of a conductive material, and the knob component 2 is rotatably connected to the rotary hole 12 and can move in an axial direction of the rotary hole 12; a head end of the knob component 2 is arranged outside; and a tail end of the knob component 2 is arranged in the accommodating chamber 10 to sleeve a periphery of the crown 32 and abut against a titanium electrode 320 on the crown 32.

Specifically, before use, a smartwatch is placed in the accommodating chamber 10, and the detection portion 31 of the watch body 3 protrudes out of the notch 11. Meanwhile, the tail end of the knob component 2 sleeves the crown 32. Finally, the watch protection case is worn on the wrist by using a strap to make the detection portion 31 abut against the wrist.

Under normal circumstances, a user can drive the crown 32 to rotate/move by turning/pressing the knob component 2 to operate the smartwatch smoothly. Since the detection portion 31 of the watch body 3 protrudes out of the notch 11, an electrical signal on the wrist can be transmitted to the detection portion 31, ensuring that the user can use a heart rate detection function of the smartwatch normally.

To use an electrocardiogram display function of the smartwatch, a user first opens relevant software on the smartwatch through the touch screen 33, and then abuts against the head end of the knob component 2 with a fingertip. Through the knob component 2, an electrical signal can be transmitted to the titanium electrode 320 of the crown 32 normally, thereby forming a signal loop between the body, the arm, and the heart, ensuring that the smartwatch can detect the heart normally and display an electrocardiogram on a touch screen 33.

It should be noted that during the use of the electrocardiogram display function, the user can also use the fingertip to press the knob component 2, so that the tail end of the knob component 2 can tightly abut against the titanium electrode 320 of the crown 32, ensuring the detection accuracy. Since the crown 32 of the smartwatch itself has a reset function, when the user releases the knob component 2, the crown 32 is reset and drives the knob component 2 to return to an initial position.

In summary, in the present disclosure, the case body 1 is designed to protect the watch body 3 and the touch screen 33 of the smartwatch to avoid the smartwatch from being damaged by collision. The notch 11 is designed on the case body 1, so that the wrist can abut against the detection portion 31 to transmit the electrical signal. The knob component 2 is designed to drive the crown 32, so that a user can operate the smartwatch. As the knob component 2 is made of the conductive material, a fingertip can transmit an electrical signal to the titanium electrode 320, causing two acquisition points of the smartwatch to form a loop to record an electrical signal of the heart. This ensures that the electrocardiogram display function can be normally used and improves the convenience of use.

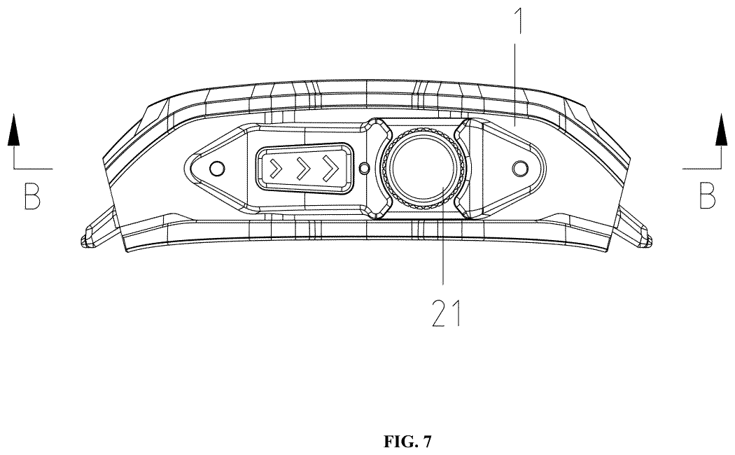

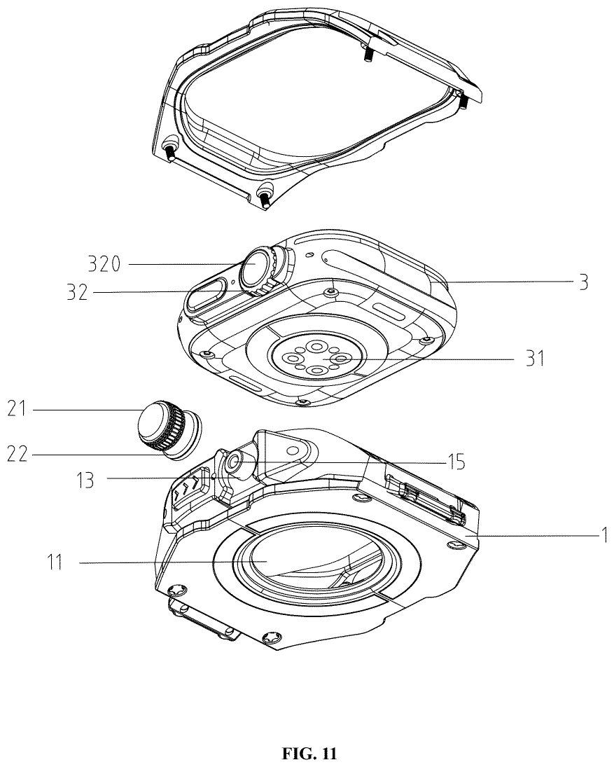

Referring to FIG. 7 to FIG. 12, the knob component 2 may be an integrally formed individual part. However, considering that it is difficult to mount the knob component on the case body 1, in this embodiment, the knob component 2 includes a wheel knob 21 and a crown sleeve 22; the crown sleeve 22 is arranged in the accommodating chamber 10 to sleeve the periphery of the crown 32 and abut against the titanium electrode 320 of the crown 32; a shaft rod 220 that is rotatably connected to the rotary hole 12 and can move in the axial direction of the rotary hole 12 is arranged on the crown sleeve 22; the wheel knob 21 is arranged outside and is connected to the shaft rod 220, so as to control rotation and movement of the crown sleeve 22; and the crown sleeve 22, the shaft rod 220, and the wheel knob 21 are all made of the conductive material.

Specifically, during mounting, the crown sleeve 22 is first placed in the accommodating chamber 10, and the shaft rod 220 penetrates through the rotary hole 12. Next, the wheel knob 21 is arranged outside and is connected to the shaft rod 220, so that the entire knob component 2 can be mounted on the case body 1. At this point, the smartwatch is placed in the accommodating chamber 10, so that the crown 32 is aligned with the crown sleeve 22. Then, the wheel knob 21 is pushed to drive the shaft rod 220 to move inwards in the axial direction of the rotary hole 12, so that the crown sleeve 22 sleeves the periphery of the crown 32 and abuts against the titanium electrode 320 on the crown 32.

During use, turning the wheel knob 21 can drive the shaft rod 220 and the crown sleeve 22, thereby achieving a turning operation on the crown 32. Pressing the wheel knob 21 can drive the shaft rod 220 and the crown sleeve 22 to move inwards, thereby achieving a pressing operation on the crown 32. After the wheel knob 21 is released, the crown 32 is reset and drives the crown sleeve 22 and the shaft rod 220 to move outwards, so that the wheel knob 21 is reset for a next operation. Since the crown sleeve 22, the shaft rod 220, and the wheel knob 21 are all made of the conductive material, when a fingertip is in contact with the wheel knob 21, an electrical signal can pass through the wheel knob 21, the shaft rod 220, and the crown sleeve 22 in sequence and be transmitted to the titanium metal on the crown 32.

In summary, due to the cooperative use of the crown sleeve 22, the shaft rod 220, and the wheel knob 21, it is convenient to assemble and disassemble the knob component 2. In addition, to prevent the shaft rod 220 from being separated from the rotary hole 12, a diameter of the crown sleeve 22 is greater than a diameter of the rotary hole 12, thereby limiting an actual outward movement range of the shaft rod 220. An inward movement range of the shaft rod 220 is generally determined by a motion distance of the crown 32 itself.

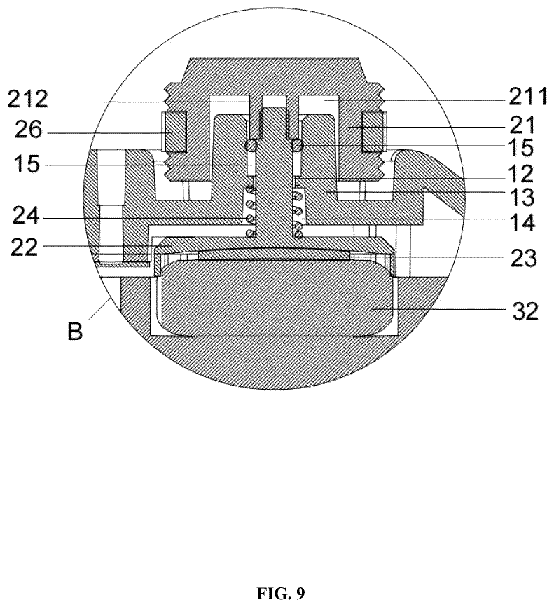

Referring to FIG. 9 to FIG. 11, an outer wall of the case body 1 is provided with a convex column 13; the rotary hole 12 is coaxially arranged in the convex column 13; the wheel knob 21 is provided with a concave cavity 211; the wheel knob 21 is coaxially provided with an integrally formed sleeve 212 in the concave cavity 211; the concave cavity 211 is matched with the convex column 13; and the sleeve 212 sleeves and fixes the shaft rod 220.

Specifically, the cooperation design of the convex column 13 and the concave cavity 211 can narrow a mounting space, to make the wheel knob 21 mounted more compactly, and can also limit a deviation of the wheel knob 21 and improve the mounting stability of the wheel knob 21. The cooperation design between the sleeve 212 and the shaft rod 220 facilitates the connection between the wheel knob 21 and the shaft rod 220 for electrical signal transmission. Moreover, the sleeve 212 and the shaft rod 220 can also be in interference fit to facilitate the mounting and removal of the wheel knob 21 and avoid easy separation of the wheel knob 21.

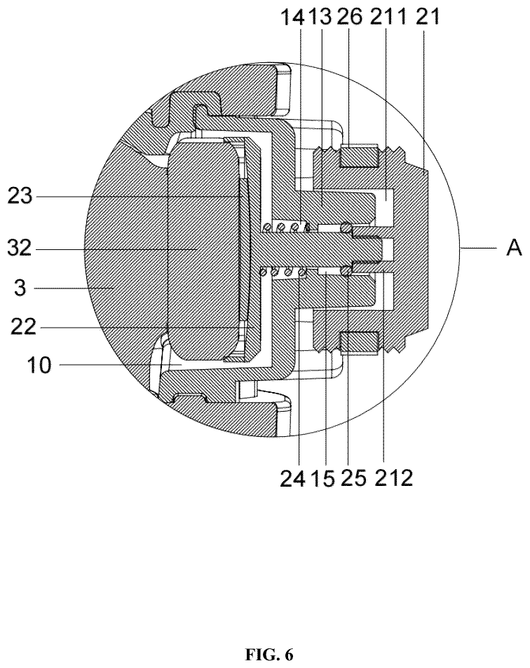

Referring to FIG. 6 and FIG. 9 to FIG. 11, the knob component 2 further includes a flexible contact block 23; the flexible contact block 23 is in a plane design and is mounted in the crown sleeve 22; the crown sleeve 22 tightly abuts against the titanium electrode 320 of the crown 32 through the flexible contact block 23; and the flexible contact block 23 is also made of a conductive material. The flexible contact block 23 in the planar design can enlarge a contact area between the crown sleeve 22 and the titanium electrode 320 of the crown 32, so as to achieve a close fit between the crown sleeve and the titanium electrode and improve the conductivity. A buffer can also be formed between the crown sleeve 22 and the crown 32 to avoid rigid collision between the crown sleeve and the crown and reduce the possibility of damage to the crown 32.

Referring to FIG. 9 to FIG. 12, there are various types of conductive materials. The crown sleeve 22, the shaft rod 220, and the wheel knob 21 are all made of a metal conductive material such as copper, iron, and 316 stainless steel to cause the crown sleeve 22, the shaft rod 220, and the wheel knob 21 to have good hardness to ensure the structural stability, thereby improving operability. The flexible contact block 23 is made of a flexible conductive material such as conductive foam, a conductive adhesive, and conductive cloth, so that the flexible contact block 23 has good deformation ability to adapt to the crown 32, ensuring a stable fit between the crown sleeve 22 and the crown 32 and improving the conductivity. Meanwhile, the flexible contact block 23 has certain buffering ability to avoid rigid collision between the crown sleeve 22 and the crown 32, thereby prolonging the service life of the crown 32.

Referring to FIG. 6 and FIG. 9 to FIG. 12, due to the sleeve connection between the crown sleeve 22 and the crown 32, there may be a gap between an inner wall surface of the crown sleeve 22 and the titanium electrode 320 of the crown 32, so that the crown sleeve and the crown cannot be in close fit. Therefore, during the use of the electrocardiogram display function, a user needs to continuously press the wheel knob 21 with a fingertip to drive the crown sleeve 22 to be in close fit with the titanium electrode 320 of the crown 32. The entire process is complicated, and due to the difficulty in controlling a pressure, it is easy to drive the crown 32 during pressing, causing electrocardiogram function software to be forced to exit and affecting the normal use. To solve this problem, in the present disclosure, the knob component 2 further includes an elastic body 24; the case body 1 is provided with a driving chamber 14 on an inner side of the rotary hole 12; the shaft rod 220 can penetrate through the driving chamber 14 and the rotary hole 12 in sequence; and the elastic body 24 is arranged in the driving chamber 14 and resists against the crown sleeve 22 and a top wall of the driving chamber 14.

Specifically, under normal circumstances, the elastic body 24 is in an initial state or a semi-compressed state to continuously provide a support for the crown sleeve 22, thereby driving the crown sleeve 22 to continuously tightly abut against the titanium electrode 320 of the crown 32. In this case, a user only needs to abut against the wheel knob 21 with a fingertip to use the electrocardiogram display function normally, thereby improving the convenience of use. Due to the fact that an elastic force of the elastic body 24 can be selected, a manufacturer can provide a fixed supporting force for the crown 22 by selecting the corresponding elastic body 24, thereby effectively preventing an excessive force from being applied to the crown 22 to drive the crown 32 to move, and ensuring that the electrocardiogram display function of the smartwatch can be used normally.

During assembling, the elastic body is first placed in the driving chamber 14 or sleeves the shaft rod 220; the shaft rod 220 then penetrates through the driving chamber 14 and the rotary hole 12 in sequence; and the wheel knob 21 located outside is then connected to the shaft rod 220. As for a use state of the elastic body 24, generally, before the smartwatch is placed in the case body 1, the elastic body 24 is in the initial state. In the process of mounting the watch body 3 of the smartwatch in the accommodating chamber 10, under the compression of the crown 32, the elastic body 24 may be compressed to store energy. Until the crown sleeve 22 is aligned with and coaxially sleeves the crown 32, the elastic body releases the energy and returns to the initial state or is in the semi-compressed state, so that the crown sleeve 22 tightly resists against the titanium electrode 320 of the crown 32.

It should be noted that under normal circumstances, the selected elastic body 24 directly returns to the initial state to support the crown sleeve 22 after the crown sleeve 22 sleeves the crown 32. However, considering that the elasticity of the elastic body 24 may be reduced after long-time use, causing a decrease in the supporting force and making it hard to continue to provide the support for the crown sleeve 22, the elastic body 24 may also return to the semi-compressed state to support the crown sleeve 22 after the crown sleeve 22 sleeves the crown 32, thereby prolonging the probable life of the elastic body 24, but it needs to ensure that the supporting force provided by the elastic body 24 in the semi-compressed state is not greater than an elastic force when the crown 32 is reset itself, so as to avoid the elastic body 24 from driving, through the crown sleeve 22, the crown 32 to move.

Further, there are various types of elastic bodies 24. For example, a spring, a rubber sleeve, and the like can be used. For ease of understanding, the elastic body 24 in this embodiment uses a spring. The spring sleeves the shaft rod 220, and two ends of the spring respectively resist against the crown sleeve 22 and the top wall of the driving chamber 14.

Referring to FIG. 9 to FIG. 12, the knob component 2 further includes a sealing ring 25; the case body 1 uses a sealed waterproof design; the case body 1 is provided with a sealing chamber 15 on an outer side of the rotary hole 12; the shaft rod 220 can penetrate through the rotary hole 12 and the sealing chamber 15 in sequence; the sealing ring 25 is arranged in the sealing chamber 15 and sleeves the shaft rod 220; and the sealing ring 25 resists and abuts against the shaft rod 220 and an inner wall of the sealing chamber 15.

Specifically, during assembling, the shaft rod 220 penetrates through the rotary hole 12 into the sealing chamber 15, and the sealing ring 25 is then sleeved in the sealing chamber 15 and onto the shaft rod 220, so that the sealing ring 25 resists against the shaft rod 220 and the inner wall of the sealing chamber 15 to achieve sealing, thereby effectively preventing external water from entering the accommodating chamber 10 through the rotary hole 12. Therefore, due to the cooperative use of the sealing chamber 15 and the sealing ring 25, the knob component 2 has a waterproof function.

It should be noted that the watch protection case with the waterproof function belongs to the existing product and is relatively common in the technical field. Therefore, the present disclosure does not provide a detailed explanation on how the case body 1 achieves waterproofing. However, regarding the waterproofing of the knob component 2, the solutions described in the prior art cannot be applied to this solution. Therefore, the waterproofing of the knob component 2 is separately designed and elaborated in the present disclosure.

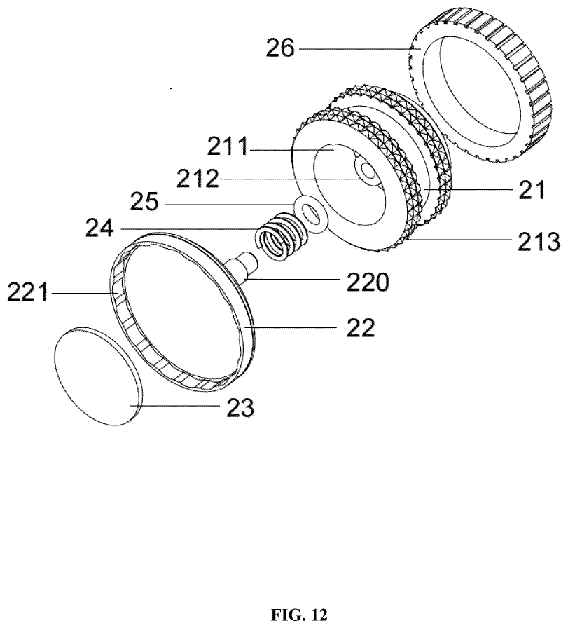

Referring to FIG. 12, a plurality of anti-slip slots 221 are arranged in a circular array on an inner wall of the crown sleeve 22 to be in close fit with a peripheral side wall of the crown 32. This design can increase a friction force between the crown sleeve 22 and the crown 32, making the connection between the crown sleeve 22 and the crown 32 more stable.

Referring to FIG. 12, skidproof stripes 213 are arranged on a circumferential side wall of the wheel knob 21. This design can provide an acting point for the wheel knob 21 to increase a friction force between the wheel knob 21 and the fingers and facilitate a user to turn the wheel knob 21.

Referring to FIG. 9 to FIG. 12, the knob component 2 further includes a decorative ring 26. The decorative ring 26 is mounted on the wheel knob 21 in a sleeving manner. The decorative ring 26 can improve the aesthetics and can be replaced by a user according to a need, thereby increasing the fun.

The above only describes the preferred embodiments of the present disclosure and is not intended to limit the present disclosure. Any modification, equivalent replacement, and improvement made within the spirit and principle of the present disclosure shall fall within the protection scope of the present disclosure.

What is claimed is:

1. A watch protection case for assisting in displaying an electrocardiogram, comprising:
a case body, wherein an accommodating chamber for placing a watch body of a watch is arranged inside the case body; a notch where a detection portion of the watch body protrudes out is arranged on a bottom wall of the accommodating chamber; a rotary hole corresponding to a crown is formed in a side wall of the accommodating chamber;
a knob component, wherein the knob component is made of a conductive material, and the knob component is rotatably connected to the rotary hole and is able to move in an axial direction of the rotary hole; a head end of the knob component is arranged outside; and a tail end of the knob component is arranged in the accommodating chamber to sleeve a periphery of the crown and abut against a titanium electrode on the crown.

2. The watch protection case for assisting in displaying the electrocardiogram according to claim 1, wherein the knob component comprises a wheel knob and a crown sleeve; the crown sleeve is arranged in the accommodating chamber to sleeve the periphery of the crown and abut against the titanium electrode of the crown; a shaft rod that is rotatably connected to the rotary hole and is able to move in the axial direction of the rotary hole is arranged on the crown sleeve; the wheel knob is arranged outside and is connected to the shaft rod, so as to control rotation and movement of the crown sleeve; and the crown sleeve, the shaft rod, and the wheel knob are all made of the conductive material.

3. The watch protection case for assisting in displaying the electrocardiogram according to claim 2, wherein an outer wall of the case body is provided with a convex column; the rotary hole is coaxially arranged in the convex column; the wheel knob is provided with a concave cavity; the wheel knob is coaxially provided with an integrally formed sleeve in the concave cavity; the concave cavity is matched with the convex column; and the sleeve sleeves and fixes the shaft rod.

4. The watch protection case for assisting in displaying the electrocardiogram according to claim 2, wherein the knob component further comprises a flexible contact block; the flexible contact block is in a plane design and is mounted in the crown sleeve; the crown sleeve tightly abuts against the titanium electrode of the crown through the flexible contact block; and the flexible contact block is also made of a conductive material.

5. The watch protection case for assisting in displaying the electrocardiogram according to claim 4, wherein the crown sleeve, the shaft rod, and the wheel knob are all made of a metal conductive material, and the flexible contact block is made of a flexible conductive material.

6. The watch protection case for assisting in displaying the electrocardiogram according to claim 2, wherein the knob component further comprises an elastic body; the case body is provided with a driving chamber on an inner side of the rotary hole; the shaft rod is able to penetrate through the driving chamber and the rotary hole in sequence; and the elastic body is arranged in the driving chamber and resists against the crown sleeve and a top wall of the driving chamber to drive the crown sleeve to tightly abut against the titanium electrode of the crown.

7. The watch protection case for assisting in displaying the electrocardiogram according to claim 6, wherein the elastic body is a spring; the spring sleeves the shaft rod; and the two ends of the spring respectively resist against the crown sleeve and the top wall of the driving chamber.

8. The watch protection case for assisting in displaying the electrocardiogram according to claim 2, wherein the knob component further comprises a sealing ring; the case body uses a sealed waterproof design; the case body is provided with a sealing chamber on an outer side of the rotary hole; the shaft rod is able to penetrate through the rotary hole and the sealing chamber in sequence; the sealing ring is arranged in the sealing chamber and sleeves the shaft rod; and the sealing ring resists and abuts against the shaft rod and an inner wall of the sealing chamber.

9. The watch protection case for assisting in displaying the electrocardiogram according to claim 2, wherein a plurality of anti-slip slots are arranged in a circular array on an inner wall of the crown sleeve to be tightly matched with a peripheral side wall of the crown.

10. The watch protection case for assisting in displaying the electrocardiogram according to claim 2, wherein skidproof stripes are arranged on a circumferential side wall of the wheel knob.
